(19) United States
(12) Patent Application Publication
(10) Pub. No.: US 2024/0226479 A1
(43) Pub. Date: Jul. 11, 2024

(54) DUAL-BALLOON VENTILATION TUBE

(71) Applicant: AIRWAY MEDIX S.A., Warszawa (PL)

(72) Inventor: Oron ZACHAR, Tel Aviv (IL)

(21) Appl. No.: 18/560,813

(22) PCT Filed: Sep. 13, 2021
(86) PCT No.: PCT/IL2021/051108

§ 371 Date: Nov. 14, 2023

Related U.S. Application Data
(60) Provisional application No. 63/079,628, filed on Sep. 17, 2020.
(60) Provisional application No. 63/188,496, filed on May 14, 2021.

(51) Int. Cl. A61M 16/04

(52) U.S. Cl. CPC A61M 16/0409; A61M 16/044

(57) ABSTRACT

A dual-balloon ventilation tube (20) includes a laryngeal ventilation support (34) supported by a distal end (54) of a shaft (50), defining a ventilation port (36), and having a greatest lateral width of 2 to 6 cm, measured between lateral sides (44) of the laryngeal ventilation support (34), such that both of the lateral sides (44) contact laryngeal soft tissue of the pharynx so as to provide lateral support that centers and inhibits tilting of the laryngeal ventilation support (34). An airway tube (56) passes through the shaft (50) and has a distal end (60) that is in fluid communication with the ventilation port (36). A proximal pharyngeal inflatable balloon (30) surrounds a distal portion (62) of the shaft (50) proximal to the laryngeal ventilation support (34), and is configured to make an air-tight seal with the pharynx. A distal esophageal inflatable balloon (32) is disposed distal to the laryngeal ventilation support (34), and is configured to make an air-tight seal with the esophagus. Other embodiments are also described.

### CROSS-REFERENCE TO RELATED APPLICATIONS

The present application claims priority from U.S. Provisional Application 63/079,628, filed Sep. 17, 2020, and U.S. Provisional Application 63/188,496, filed May 14, 2021, all of which are assigned to the assignee of the present application and incorporated herein by reference.

### FIELD OF THE APPLICATION

The present invention relates generally to lung ventilation devices, and specifically to ventilation tubes.

### BACKGROUND OF THE APPLICATION

Laryngeal tubes are designed to secure a patient airway during anesthesia and cardiopulmonary resuscitation. A laryngeal tube may include a primary inflatable cuff, mask, or elastomeric cuff disposed on its distal end for blocking the esophagus, and a more proximal secondary inflatable or elastomeric barrier for blocking the pharynx. The laryngeal tube further includes a channel to establish fluid communication with the larynx.

### SUMMARY OF THE APPLICATION

In some embodiments of the present invention, a dual-balloon ventilation tube is provided that comprises a laryngeal ventilation support, which is shaped so as to define a ventilation port, which is configured to be in fluid communication with a laryngeal inlet of a patient when the laryngeal ventilation support is disposed at an insertion location within a larynx of the patient. Typically, the laryngeal ventilation support is ergonomically configured such that when the laryngeal ventilation support is disposed at the insertion location, both of the lateral sides contact laryngeal soft tissue of the larynx so as to provide lateral support that centers and inhibits tilting of the laryngeal ventilation support, such that the ventilation port faces anteriorly. Typically, the laryngeal ventilation support has a greatest lateral width of between 2 and 6 cm, measured between lateral sides of the laryngeal ventilation support.

The dual-balloon ventilation tube further comprises a shaft having proximal and distal ends. The proximal end is configured to be disposed outside an oral cavity of the patient when the laryngeal ventilation support is disposed at the insertion location, and the distal end supports the laryngeal ventilation support.

The dual-balloon ventilation tube further comprises an airway tube, which passes through at least a longitudinal portion of the shaft, and has a proximal end that is configured to be disposed outside the patient's oral cavity when the laryngeal ventilation support is disposed at the insertion location, and a distal end that is in fluid communication with the ventilation port.

The dual-balloon ventilation tube further comprises a proximal pharyngeal inflatable balloon, which surrounds a distal portion of the shaft proximal to the laryngeal ventilation support, and which is configured to make an air-tight seal with a pharynx of the patient when the laryngeal ventilation support is disposed at the insertion location.

The dual-balloon ventilation tube further comprises a distal esophageal inflatable balloon, which is disposed distal to the laryngeal ventilation support, and which is configured to make an air-tight seal with an esophagus of the patient when the laryngeal ventilation support is disposed at the insertion location.

There is therefore provided, in accordance with an application of the present invention, a dual-balloon ventilation tube including:
a laryngeal ventilation support, which:
   is shaped so as to define a ventilation port, which is configured to be in fluid communication with a laryngeal inlet of a patient when the laryngeal ventilation support is disposed at an insertion location within a pharynx of the patient, and
   has a greatest lateral width of between 2 and 6 cm, measured between lateral sides of the laryngeal ventilation support, such that when the laryngeal ventilation support is disposed at the insertion location, both of the lateral sides contact laryngeal soft tissue of the pharynx so as to provide lateral support that centers and inhibits tilting of the laryngeal ventilation support, such that the ventilation port faces anteriorly;
a shaft having proximal and distal ends, the proximal end configured to be disposed outside an oral cavity of the patient when the laryngeal ventilation support is disposed at the insertion location, and the distal end supporting the laryngeal ventilation support;
an airway tube, which passes through at least a longitudinal portion of the shaft, and has a proximal end that is configured to be disposed outside the patient's oral cavity when the laryngeal ventilation support is disposed at the insertion location, and a distal end that is in fluid communication with the ventilation port;
a proximal pharyngeal inflatable balloon, which surrounds a distal portion of the shaft proximal to the laryngeal ventilation support, and which is configured to make an air-tight seal with a pharynx of the patient when the laryngeal ventilation support is disposed at the insertion location; and
a distal esophageal inflatable balloon, which is disposed distal to the laryngeal ventilation support, and which is configured to make an air-tight seal with an esophagus of the patient when the laryngeal ventilation support is disposed at the insertion location.

For some applications, the greatest lateral width of the laryngeal ventilation support is between 3 and 6 cm.

For some applications, the laryngeal ventilation support, along a 1-cm-long longitudinal portion thereof, has an average lateral width greater than 90% of the greatest lateral width of the laryngeal ventilation support.

For some applications, the greatest lateral width of the laryngeal ventilation support is at least 1.5 times a greatest anterior-posterior height of the laryngeal ventilation support.

For some applications, the greatest lateral width of the laryngeal ventilation support is at least 2 times the greatest anterior-posterior height of the laryngeal ventilation support.

For some applications, respective portions of a perimeter of the laryngeal ventilation support defined by the lateral sides are reflectionally symmetric with each other.

For some applications:
a central longitudinal axis of the dual-balloon ventilation tube is defined by a line that passes through a centroid of the distal esophageal balloon and a centroid of the proximal pharyngeal inflatable balloon,
a projection of the laryngeal ventilation support onto a plane, in which the central longitudinal axis falls, is divided by the central longitudinal axis into two lateral sections on either side of the central longitudinal axis 88, and
an area of one of the lateral sections includes between 40% and 50% of a total area of the projection.

For some applications, the area of the one of the lateral sections includes between 45% and 50% of the total area of the projection.

For some applications, the laryngeal ventilation support is shaped so as to define an anterior side that is configured to stabilize the laryngeal ventilation support with respect to the laryngeal inlet when the laryngeal ventilation support is disposed at the insertion location.

For some applications, the proximal pharyngeal balloon, when unconstrained and inflated at a pressure of 5 cm H2O, has a greatest lateral width that is between 1.2 and 2 times the greatest lateral width of the laryngeal ventilation support.

For some applications, the proximal pharyngeal balloon, when unconstrained and inflated at a pressure of 5 cm H2O, has a greatest anterior-posterior height that is between 1 and 2 times a greatest anterior-posterior height of the laryngeal ventilation support.

For some applications:
the proximal pharyngeal balloon has a higher-pressure aspect ratio equal to a greatest lateral width of the proximal pharyngeal balloon divided by a greatest anterior-posterior height of the proximal pharyngeal balloon when the proximal pharyngeal balloon is unconstrained and inflated at a pressure of 30 cm H2O,
the proximal pharyngeal balloon has a lower-pressure aspect ratio equal to a greatest lateral width of the proximal pharyngeal balloon divided by a greatest anterior-posterior height of the proximal pharyngeal balloon when the proximal pharyngeal balloon is unconstrained and inflated at a pressure of 5 cm H2O, and
the higher-pressure aspect ratio is less than the lower-pressure aspect ratio.

For some applications, the higher-pressure aspect ratio is less than 85% of the lower-pressure aspect ratio.

For some applications, the proximal pharyngeal balloon, when unconstrained and inflated at a pressure of 5 cc H2O, has a greatest lateral width that is between 1.2 and 2 times a lateral width of the shaft at an interface between the shaft and a proximal end of the proximal pharyngeal balloon.

For some applications, the proximal pharyngeal balloon, when unconstrained and inflated at a pressure of 5 cc H2O, has a greatest anterior-posterior distance from the shaft that is between 0 and 5 mm.

For some applications, the proximal pharyngeal balloon, when unconstrained and inflated at a pressure of 5 cc H2O, has:
a greatest lateral width that is between 1.2 and 2 times a lateral width of the shaft at an interface between the shaft and a proximal end of the proximal pharyngeal balloon, and
a greatest anterior-posterior distance from the shaft that is between 0 and 5 mm.

For some applications, the proximal pharyngeal balloon, when unconstrained and inflated at a pressure of 5 cc H2O, has a greatest lateral distance from a lateral edge of the shaft that is between 5 and 10 mm, and has a greatest anterior-posterior distance from the shaft that is between 0 and 5 mm.

For some applications, the lateral sides of the laryngeal ventilation support are shaped so as to define respective wings, which are configured to flex toward a central longitudinal axis of the laryngeal ventilation support when squeezed by the laryngeal soft tissue when the laryngeal ventilation support is disposed at the insertion location.

For some applications, the proximal pharyngeal balloon is generally ellipsoidal when unconstrained and inflated at a pressure of 5 cc H2O.

For some applications, the laryngeal ventilation support includes a material having a Shore hardness of between A30 and A90.

For some applications, the proximal pharyngeal balloon, when unconstrained and inflated at a pressure of 5 cm H2O, is shaped such that:
the proximal pharyngeal balloon has greatest lateral width at a location along the proximal pharyngeal balloon,
a line segment having the greatest lateral width extends between opposing lateral sides of the proximal pharyngeal balloon at the location,
the proximal pharyngeal balloon has a greatest-width cross-section perpendicular to a central longitudinal axis of the laryngeal ventilation support, and an outer perimeter of the greatest-width cross-section is divided by the line segment into a posterior perimeter portion and an anterior perimeter portion, and
a ratio of a length of the posterior perimeter portion to a length of the anterior perimeter portion is between 1.2:1 and 2:1, the lengths measured along the respective perimeter portions.

For some applications, the proximal pharyngeal balloon, when unconstrained and inflated at a pressure of 5 cm H2O, is shaped such that:
the proximal pharyngeal balloon has greatest lateral width at a location along the proximal pharyngeal balloon,
a line segment having the greatest lateral width connects lateral sides of the proximal pharyngeal balloon at the location,
the proximal pharyngeal balloon has a greatest-width cross-section perpendicular to a central longitudinal axis of the laryngeal ventilation support, and
a ratio of (a) a first greatest distance, in the greatest-width cross-section, between a posterior side of the proximal pharyngeal balloon and the line segment to (b) a second greatest distance, in the greatest-width cross-section, between an anterior side of the proximal pharyngeal balloon and the line segment, is between 1.2:1 and 2:1.

For some applications, the proximal pharyngeal balloon, when unconstrained and inflated at a pressure of 5 cm H2O, is shaped such that:
the proximal pharyngeal balloon has greatest lateral width at a location along the proximal pharyngeal balloon,
a line segment having the greatest lateral width connects lateral sides of the proximal pharyngeal balloon at the location,
the proximal pharyngeal balloon has a greatest-width cross-section perpendicular to a central longitudinal axis of the laryngeal ventilation support,
the greatest-width cross-section is divided by the line segment into a posterior cross-sectional portion and an anterior cross-sectional portion, and
a ratio of an area of the posterior cross-sectional portion to an area of the anterior cross-sectional portion is between 1.2:1 and 2:1.

For some applications:
the dual-balloon ventilation tube further comprises an externally-accessible inflation tube, which has a distal end that is coupled in fluid communication with an interior of the proximal pharyngeal inflatable balloon, and
the dual-balloon ventilation tube is configured such that when a volume of 6 cc of ambient air, the volume measured at standard atmospheric pressure, is introduced into the inflation tube while the proximal pharyngeal balloon and the distal esophageal inflatable balloon are unconstrained, disposed in ambient air that is at atmospheric pressure, and is at an inflation pressure of 30 cm H2O:
   the inflation pressure in the proximal pharyngeal balloon, measured one minute after introduction of the volume of 6 cc of the ambient air, is less than 40 cm H2O.

For some applications, a posterior surface of the laryngeal ventilation support is shaped so as to define at least three elongate grooves.

For some applications, the laryngeal ventilation support is not inflatable.

For some applications, at least 80% of an external surface of the laryngeal ventilation support is not covered by any inflatable balloon.

For some applications:
the dual-balloon ventilation tube further includes an externally-accessible inflation tube, which (a) includes an inflation valve that is configured to be disposed outside the patient's oral cavity when the laryngeal ventilation support is disposed at the insertion location, and (b) has a distal end that is coupled in fluid communication with an interior of the proximal pharyngeal inflatable balloon, and
the dual-balloon ventilation tube is configured such that when the laryngeal ventilation support, while the inflation valve is closed and a pressure within the proximal pharyngeal inflatable balloon equals ambient pressure, is inserted from outside the oral cavity of the patient to the insertion location without opening the inflation valve, the pressure within the proximal pharyngeal inflatable balloon increases from the ambient pressure to an inserted-location pressure, the inserted-location pressure less than 60 cm H2O.

For some applications, the inserted-location pressure is less than 45 cm H2O.

For some applications, the inserted-location pressure is less than 30 cm H2O.

For some applications, the proximal pharyngeal inflatable balloon and the distal esophageal inflatable balloon are connected in fluid communication.

For some applications, a ventilation tube system is provided that includes the dual-balloon ventilation tube and further includes an insertion posterior protector, which includes an elongate sheet of material that is shaped so as to define:
a proximal coupling portion, which is shaped so as to define one or more couplers that are configured to removably couple the proximal coupling portion to the shaft proximally to the proximal pharyngeal balloon, and
a distal protection portion, which is shaped so as to partially cover a posterior external surface of the proximal pharyngeal inflatable balloon when the proximal coupling portion is removably coupled to the shaft, so as to protect the proximal pharyngeal balloon from puncture during insertion thereof into the patient's oral cavity.

For some applications, the distal protection portion is shaped so as to additionally partially cover a posterior external surface of the distal esophageal balloon when the proximal coupling portion is removably coupled to the shaft, so as to protect the distal esophageal balloon from puncture during insertion thereof into the patient's oral cavity.

For some applications, the one or more couplers are shaped so as to form snap-on couplings to the shaft.

For some applications, the elongate sheet of material of the insertion posterior protector is further shaped so as to define a proximal handle, which is shaped so as to be disposed outside the patient's oral cavity when the laryngeal ventilation support is disposed at the insertion location.

There is further provided, in accordance with an application of the present invention, a method including:
inserting a distal esophageal inflatable balloon, a laryngeal ventilation support, and a proximal pharyngeal inflatable balloon of a dual-balloon ventilation tube through an anterior opening of an oral cavity of a patient, such that:
   a proximal end of a shaft is disposed outside the oral cavity, wherein the distal end of the shaft supports the laryngeal ventilation support,
   a ventilation port of the laryngeal ventilation support is in fluid communication with a laryngeal inlet of the patient, and the laryngeal ventilation support is disposed at an insertion location within a pharynx of the patient such that both of lateral sides of the laryngeal ventilation support contact laryngeal soft tissue of the pharynx so as to provide lateral support that centers and inhibits tilting of the laryngeal ventilation support, such that the ventilation port faces anteriorly,
   a proximal end of an airway tube is disposed outside the patient's oral cavity, wherein the airway tube passes through at least a longitudinal portion of the shaft, and has a distal end that is in fluid communication with the ventilation port,
   the proximal pharyngeal inflatable balloon makes an air-tight seal with a pharynx of the patient, wherein the proximal pharyngeal inflatable balloon surrounds a distal portion of the shaft proximal to the laryngeal ventilation support, and
   the distal esophageal inflatable balloon makes an air-tight seal with an esophagus of the patient, wherein the distal esophageal inflatable balloon is disposed distal to the laryngeal ventilation support; and ventilating lungs of the patient using the dual-balloon ventilation tube.

For some applications, the laryngeal ventilation support has a greatest lateral width of between 2 and 6 cm, measured between lateral sides of the laryngeal ventilation support.

For some applications:
inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon while:
   a proximal coupling portion of an elongate sheet of material of an insertion posterior protector is removably coupled to the shaft proximally to the proximal pharyngeal inflatable balloon by one or more couplers of the proximal coupling portion, and
   a distal protection portion of the elongate sheet of material partially covers a posterior external surface of the proximal pharyngeal balloon, so as to protect the proximal pharyngeal inflatable balloon from puncture during insertion thereof into the patient's oral cavity, and
the method further includes, after inserting the distal esophageal inflatable balloon, the laryngeal ventilation support, and the proximal pharyngeal inflatable balloon, decoupling the insertion posterior protector from the shaft and removing the insertion posterior protector from the patient's oral cavity while the laryngeal ventilation support remains disposed at the insertion location.

For some applications, inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon while the distal protection portion additionally partially covers a posterior external surface of the distal esophageal inflatable balloon, so as to protect the distal esophageal inflatable balloon from puncture during insertion thereof into the patient's oral cavity.

For some applications, the one or more couplers are shaped so as to form snap-on couplings to the shaft.

For some applications, inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon using a proximal handle of the elongate sheet of material, while the proximal handle is disposed outside the patient's oral cavity.

For some applications:
inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon such that an inflation valve of an inflation tube of the dual-balloon ventilation tube is disposed outside the patient's oral cavity, a distal end of the inflation tube coupled in fluid communication with an interior of the proximal pharyngeal inflatable balloon,
inserting the proximal pharyngeal inflatable balloon includes inserting the proximal pharyngeal inflatable balloon, while the inflation valve is closed, from outside the oral cavity of the patient until the laryngeal ventilation support is disposed at the insertion location without opening the inflation valve, and
the dual-balloon ventilation tube is configured such that when the laryngeal ventilation support, while the inflation valve is closed and a pressure within the proximal pharyngeal inflatable balloon equals ambient pressure, is inserted from outside the oral cavity of the patient to the insertion location without opening the inflation valve, the pressure within the proximal pharyngeal inflatable balloon increases from the ambient pressure to an inserted-location pressure, the inserted-location pressure less than 60 cm H2O.

For some applications, inserting the proximal pharyngeal inflatable balloon while the inflation valve is closed includes inserting the proximal pharyngeal inflatable balloon while the inflation valve is closed and the pressure within the proximal pharyngeal inflatable balloon equals ambient pressure.

For some applications, inserting the proximal pharyngeal inflatable balloon while the inflation valve is closed includes inserting the proximal pharyngeal inflatable balloon while the inflation valve is closed without deflating the proximal pharyngeal inflatable balloon before inserting the proximal pharyngeal inflatable balloon.

For some applications, the method does not include measuring the pressure within the proximal pharyngeal inflatable balloon.

For some applications, the method does not include adding air to or removing air from the proximal pharyngeal inflatable balloon after inserting the laryngeal ventilation support is to the insertion location.

For some applications, the method does not include adjusting the pressure within the proximal pharyngeal inflatable balloon after inserting the laryngeal ventilation support to the insertion location.

For some applications, the inserted-location pressure is less than 45 cm H2O.

For some applications, the inserted-location pressure is less than 30 cm H2O.

There is still further provided, in accordance with an application of the present invention, a ventilation tube system including:
a dual-balloon ventilation tube including:
   a laryngeal ventilation support, which is shaped so as to define a ventilation port, which is configured to be in fluid communication with a laryngeal inlet of a patient when the laryngeal ventilation support is disposed at an insertion location within a pharynx of the patient;
   a shaft having proximal and distal ends, the proximal end configured to be disposed outside an oral cavity of the patient when the laryngeal ventilation support is disposed at the insertion location, and the distal end supporting the laryngeal ventilation support;
   an airway tube, which passes through at least a longitudinal portion of the shaft, and has a proximal end that is configured to be disposed outside the patient's oral cavity when the laryngeal ventilation support is disposed at the insertion location, and a distal end that is in fluid communication with the ventilation port;
   a proximal pharyngeal inflatable balloon, which surrounds a distal portion of the shaft proximal to the laryngeal ventilation support, and which is configured to make an air-tight seal with a pharynx of the patient when the laryngeal ventilation support is disposed at the insertion location; and
   a distal esophageal inflatable balloon, which is disposed distal to the laryngeal ventilation support, and which is configured to make an air-tight seal with an esophagus of the patient when the laryngeal ventilation support is disposed at the insertion location; and
an insertion posterior protector, which includes an elongate sheet of material that is shaped so as to define:
   a proximal coupling portion, which is shaped so as to define one or more couplers that are configured to removably couple the proximal coupling portion to the shaft proximally to the proximal pharyngeal inflatable balloon, and
   a distal protection portion, which is shaped so as to partially cover a posterior external surface of the proximal pharyngeal inflatable balloon when the proximal coupling portion is removably coupled to the shaft, so as to protect the proximal pharyngeal inflatable balloon from puncture during insertion thereof into the patient's oral cavity.

For some applications, the distal protection portion is shaped so as to additionally partially cover a posterior external surface of the distal esophageal inflatable balloon when the proximal coupling portion is removably coupled to the shaft, so as to protect the distal esophageal inflatable balloon from puncture during insertion thereof into the patient's oral cavity.

For some applications, the one or more couplers are shaped so as to form snap-on couplings to the shaft.

For some applications, the elongate sheet of material of the insertion posterior protector is further shaped so as to define a proximal handle, which is shaped so as to be disposed outside the patient's oral cavity when the laryngeal ventilation support is disposed at the insertion location.

For some applications, the proximal pharyngeal inflatable balloon and the distal esophageal inflatable balloon are connected in fluid communication.

There is additionally provided, in accordance with an application of the present invention, a method including:
inserting a distal esophageal inflatable balloon, a laryngeal ventilation support, and a proximal pharyngeal inflatable balloon of a dual-balloon ventilation tube through an anterior opening of an oral cavity of a patient, such that:
   a proximal end of a shaft is disposed outside the oral cavity, wherein the distal end of the shaft supports the laryngeal ventilation support,
   a ventilation port of the laryngeal ventilation support is in fluid communication with a laryngeal inlet of the patient, and the laryngeal ventilation support is disposed at an insertion location within a pharynx of the patient,
   a proximal end of an airway tube is disposed outside the patient's oral cavity, wherein the airway tube passes through at least a longitudinal portion of the shaft, and has a distal end that is in fluid communication with the ventilation port,
   the proximal pharyngeal inflatable balloon makes an air-tight seal with a pharynx of the patient, wherein the proximal pharyngeal inflatable balloon surrounds a distal portion of the shaft proximal to the laryngeal ventilation support, and
   the distal esophageal inflatable balloon makes an air-tight seal with an esophagus of the patient, wherein the distal esophageal inflatable balloon is disposed distal to the laryngeal ventilation support; and ventilating lungs of the patient using the dual-balloon ventilation tube,
wherein inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon while:
   a proximal coupling portion of an elongate sheet of material of an insertion posterior protector is removably coupled to the shaft proximally to the proximal pharyngeal inflatable balloon by one or more couplers of the proximal coupling portion,
   a distal protection portion of the elongate sheet of material partially covers a posterior external surface of the proximal pharyngeal inflatable balloon, so as to protect the proximal pharyngeal inflatable balloon from puncture during insertion thereof into the patient's oral cavity, and
wherein the method further includes, after inserting the distal esophageal inflatable balloon, the laryngeal ventilation support, and the proximal pharyngeal inflatable balloon such that the laryngeal ventilation support is disposed at the insertion location:
decoupling the insertion posterior protector from the shaft and removing the insertion posterior protector from the patient's oral cavity while the laryngeal ventilation support remains disposed at the insertion location.

For some applications, inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon while:
the distal protection portion is shaped so as to additionally partially cover a posterior external surface of the distal esophageal inflatable balloon, so as to protect the distal esophageal inflatable balloon from puncture during insertion thereof into the patient's oral cavity.

For some applications, the one or more couplers are shaped so as to form snap-on couplings to the shaft.

For some applications, inserting the distal esophageal inflatable balloon, the laryngeal ventilation support, and the proximal pharyngeal inflatable balloon includes inserting the distal esophageal inflatable balloon, the laryngeal ventilation support, and the proximal pharyngeal inflatable balloon includes using a proximal handle of the elongate sheet material, while the proximal handle is disposed outside the patient's oral cavity.

The present invention will be more fully understood from the following detailed description of embodiments thereof, taken together with the drawings, in which:

### BRIEF DESCRIPTION OF THE DRAWINGS

FIGS. 1A-E are schematic illustrations of several views of a dual-balloon ventilation tube, in accordance with an application of the present invention;

FIGS. 2A-B are schematic illustrations of several views of the dual-balloon ventilation tube of FIGS. 1A-E, in accordance with an application of the present invention;

FIGS. 3A-B are schematic cross-sectional views of the dual-balloon ventilation tube of FIGS. 1A-E, in accordance with an application of the present invention;

FIG. 4 is a schematic illustration of the dual-balloon ventilation tube of FIGS. 1A-E inserted through an anterior opening of an oral cavity of a patient, in accordance with an application of the present invention; and

FIGS. 5A-B are schematic illustrations of an insertion posterior protector coupled to and decoupled from the dual-balloon ventilation tube of FIGS. 1A-E, respectively, in accordance with an application of the present invention.

### DETAILED DESCRIPTION OF APPLICATIONS

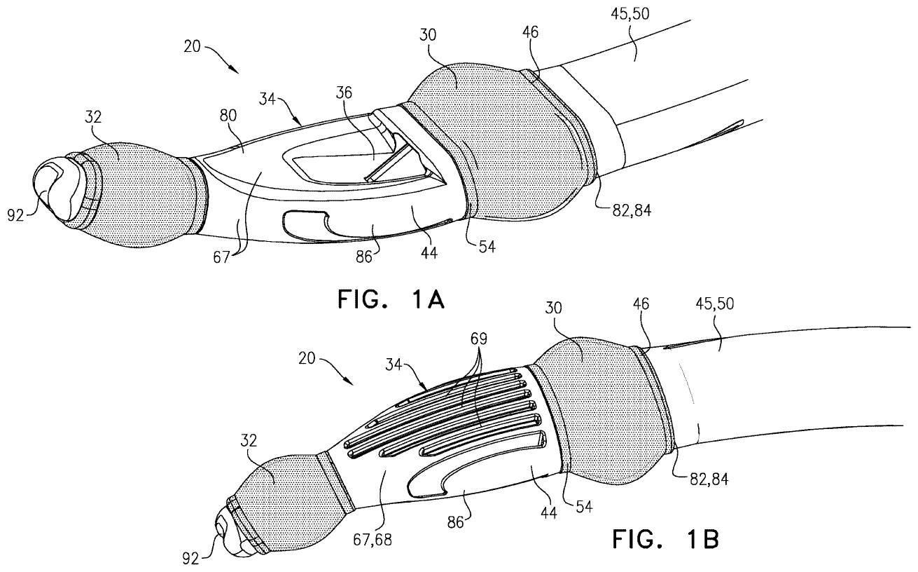

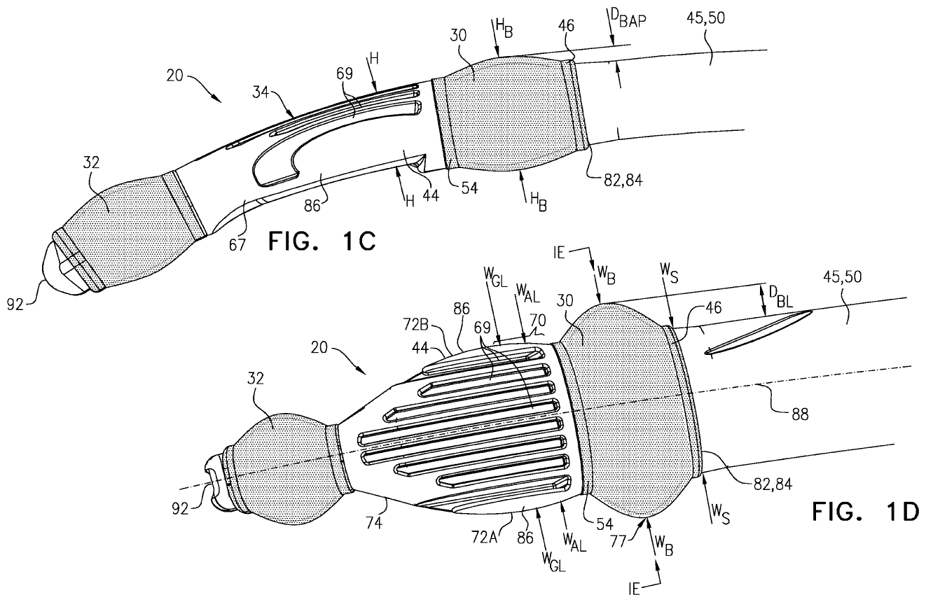

Reference is made to FIGS. 1A-E, which are schematic illustrations of several views of a dual-balloon ventilation tube 20, in accordance with an application of the present invention. FIG. 1E is a schematic cross-sectional view of dual-balloon ventilation tube 20 taken along line IE-IE of FIG. 1D.

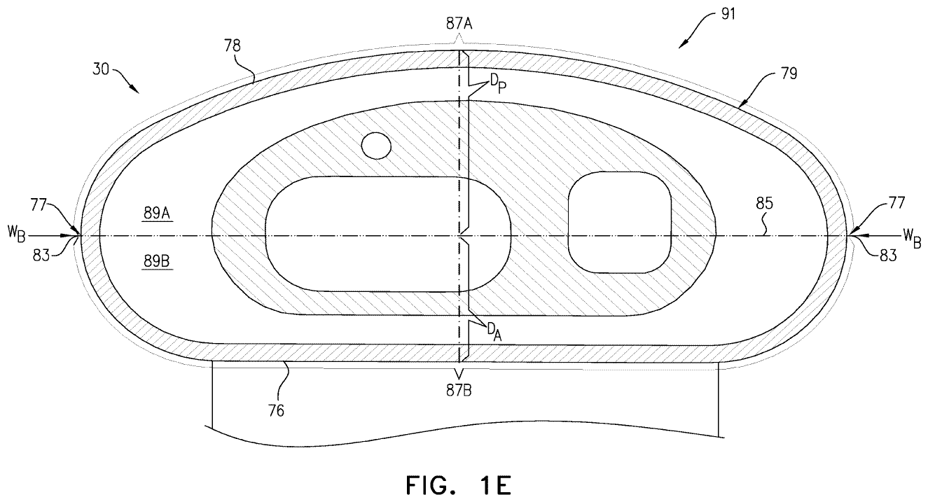

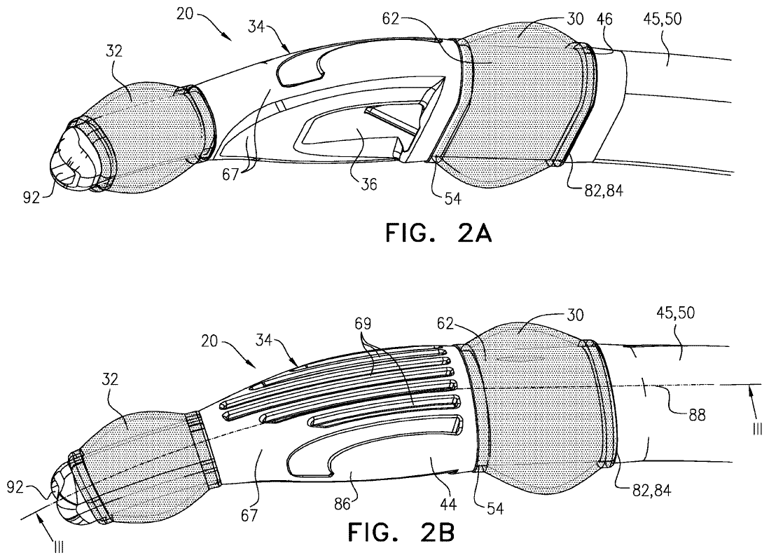

Reference is also made to FIGS. 2A-B, which are schematic illustrations of several views of dual-balloon ventilation tube 20, in accordance with an application of the present invention. In FIGS. 2A-D_{BAP}, a proximal pharyngeal inflatable balloon 30 and a distal esophageal inflatable balloon 32 of dual-balloon ventilation tube 20 are shown as translucent to show some elements the view of which would otherwise be blocked by the balloons. In practice, the balloons may be translucent, transparent, or opaque, as is known in the art.

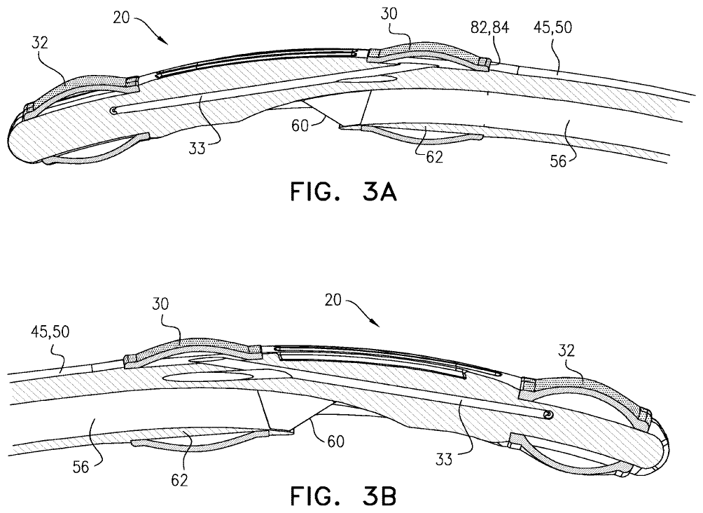

Reference is also made to FIGS. 3A-B, which are schematic cross-sectional views of dual-balloon ventilation tube 20, in accordance with an application of the present invention.

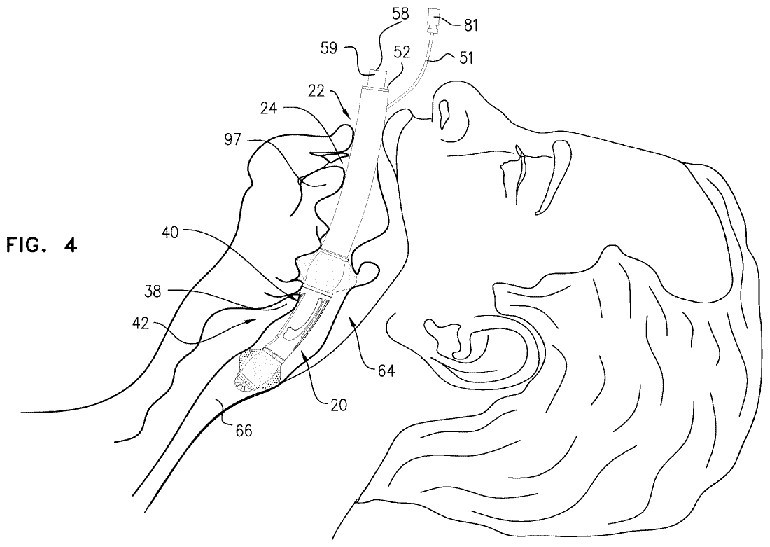

Reference is further made to FIG. 4, which is a schematic illustration of dual-balloon ventilation tube 20 inserted through an anterior opening 22 of an oral cavity 24 of a patient, in accordance with an application of the present invention. (Anterior opening 22 is the opening of the mouth between lips, between outside oral cavity 24 and inside oral cavity 24.)

Dual-balloon ventilation tube 20 comprises a laryngeal ventilation support 34, which is shaped so as to define a ventilation port 36, which is configured to be in fluid communication with a laryngeal inlet 38 of the patient so as to supply air to a larynx 42, when laryngeal ventilation support 34 is disposed at an insertion location 40 within a pharynx 64 of the patient, such as shown in FIG. 4.

Typically, laryngeal ventilation support 34 has a greatest lateral width W_{GL} (labeled in FIG. 1D) of between 2 and 6 cm, measured between lateral sides 44 of laryngeal ventilation support 34, such that when laryngeal ventilation support 34 is disposed at insertion location 40, both of lateral sides 44 contact laryngeal soft tissue of pharynx 64 so as to provide lateral support that centers and inhibits (e.g., prevents) tilting of laryngeal ventilation support 34, such that ventilation port 36 faces anteriorly. Optionally, the greatest lateral width W_{GL} of laryngeal ventilation support 34 is between 3 and 6 cm, such as between 3 and 5.5 cm. Alternatively, the greatest lateral width W_{GL} is less than 2 cm and/or greater than 6 cm.

Dual-balloon ventilation tube 20 further comprises a shaft 50 and an airway tube 56 (labeled in FIGS. 3A-B), which passes through at least a longitudinal portion of shaft 50 such as through an entirety of a length of shaft 50, as shown in the drawings. For some applications, shaft 50 is shaped, e.g., molded, so as to define at least a portion of airway tube 56 within shaft 50.

Shaft 50 has (a) a proximal end 52 that is configured to be disposed outside oral cavity 24 when laryngeal ventilation support 34 is disposed at insertion location 40, and a distal end 54 that supports laryngeal ventilation support 34. Typically, a proximal portion of shaft 50 is disposed outside the patient's oral cavity 24 when laryngeal ventilation support 34 is disposed at insertion location 40, so as to serve as a handle 45 for inserting dual-balloon ventilation tube 20 into the patient's oral cavity 24 and properly positioning dual-balloon ventilation tube 20.

Airway tube 56 has (a) a proximal end 58 (labeled in FIG. 4) that is configured to be disposed outside the patient's oral cavity 24 when laryngeal ventilation support 34 is disposed at insertion location 40, and (b) a distal end 60 (labeled in FIG. 3A) that is in fluid communication with ventilation port 36. Proximal end 58 is typically defined by an airway connector port 59, which is configured for connection to air or other ventilating apparatus for the patient's lungs.

For some applications, shaft 50 comprises PVC, silicone, or TPE. For these applications, shaft 50 may optionally have a Shore A hardness of at least 30, typically between A40 and A80.

Proximal pharyngeal balloon 30 surrounds a distal portion 62 of shaft 50 (labeled in FIGS. 2A-B and 3A) proximal to laryngeal ventilation support 34, and is configured to make an air-tight seal with pharynx 64 of the patient when laryngeal ventilation support 34 is disposed at insertion location 40. Typically, proximal pharyngeal balloon 30 forms an air-tight seal 46 with an external surface of shaft 50 (such as by gluing or welding). Typically, proximal pharyngeal balloon 30 and shaft 50 are configured such that proximal pharyngeal balloon 30 forms air-tight seal 46 entirely around a longitudinal portion of shaft 50.

Distal esophageal balloon 32 is disposed distal to laryngeal ventilation support 34, and is configured to make an air-tight seal with an esophagus 66 of the patient (typically, with an upper esophageal sphincter of esophagus 66) when laryngeal ventilation support 34 is disposed at insertion location 40.

Typically, laryngeal ventilation support 34 is not inflatable.

Typically, at least 80%, e.g., at least 90%, such as 100%, of an external surface 67 of laryngeal ventilation support 34 is not covered by any inflatable balloon.

For some applications, a posterior surface 68 of laryngeal ventilation support 34 is shaped so as to define at least three, e.g., at least four, elongate grooves 69, and/or no more than ten, e.g., no more than eight elongate grooves 69. Elongate grooves 69 may reduce friction with tissue during insertion.

For some applications, laryngeal ventilation support 34, along a 1-cm-long longitudinal portion 70 thereof, has an average lateral width W_{AL} (labeled in FIG. 1D) greater than 90% of the greatest lateral width W_{GL} (labeled in FIG. 1D) of laryngeal ventilation support 34.

For some applications, the greatest lateral width W_{GL} of laryngeal ventilation support 34 is at least 1.5 times, such as at least 2 times, a greatest anterior-posterior height H (labeled in FIG. 1C) of laryngeal ventilation support 34.

For some applications, laryngeal ventilation support 34 is shaped so as to define an anterior side 80 that is configured to stabilize laryngeal ventilation support 34 with respect to laryngeal inlet 38 when laryngeal ventilation support 34 is disposed at insertion location 40.

For some applications, proximal pharyngeal balloon 30, when unconstrained and inflated at a pressure of 5 cm H2O, has a greatest lateral width W_{B} (labeled in FIG. 1D) that is between 1.2 and 2 times the greatest lateral width W_{GL} (labeled in FIG. 1D) of laryngeal ventilation support 34.

For some applications, proximal pharyngeal balloon 30, when unconstrained and inflated at a pressure of 5 cm H2O, has a greatest anterior-posterior height H_{B} (labeled in FIG. 1C) that is between 1 and 2 times the greatest anterior-posterior height H (labeled in FIG. 1C) of laryngeal ventilation support 34.

For some applications, proximal pharyngeal balloon 30 has:
a higher-pressure aspect ratio equal to the greatest lateral width W_{B} (labeled in FIG. 1D) of proximal pharyngeal balloon 30 divided by the greatest anterior-posterior height H_{B} (labeled in FIG. 1C) of proximal pharyngeal balloon 30 when proximal pharyngeal balloon 30 is unconstrained and inflated at a pressure of 30 cm H2O, and
a lower-pressure aspect ratio equal to the greatest lateral width W_{B} (labeled in FIG. 1D) of proximal pharyngeal balloon 30 divided by the greatest anterior-posterior height H_{B} (labeled in FIG. 1C) of proximal pharyngeal balloon 30 when proximal pharyngeal balloon 30 is unconstrained and inflated at a pressure of 5 cm H2O.

The higher-pressure aspect ratio is less than the lower-pressure aspect ratio, such as less than 85% of the lower-pressure aspect ratio, e.g., less than 75% or less than 65% of the lower-pressure aspect ratio. Typically, resting shape of proximal pharyngeal balloon 30 is oval. When inflated in free space, the balloon becomes much more rounded.

For some applications, proximal pharyngeal balloon 30, when unconstrained and inflated at a pressure of 5 cc H2O, has a greatest lateral width W_{B} (labeled in FIG. 1D) that is between 1.2 and 2 times a lateral width W_{S} (labeled in FIG. 1D) of shaft 50 at an interface 82 between shaft 50 and a proximal end 84 of proximal pharyngeal balloon 30.

Alternatively or additionally, for some applications, proximal pharyngeal balloon 30, when unconstrained and inflated at a pressure of 5 cc H2O, has a greatest anterior-posterior distance D_{BAP} from shaft 50 (labeled in FIG. 1C) that is between 0 and 5 mm.

For some applications, proximal pharyngeal balloon 30 comprises a highly elastic material, such as silicone, latex, or TPE, and is ergonomically pre-shaped and sized to fit the space bounded by the tongue, soft palate and/or pharyngeal wall; for example, an anterior side 76 of proximal pharyngeal balloon 30 may be generally flat, and a posterior side 78 of proximal pharyngeal balloon 30 may be curved. The ergonomic fit reduces a number of folds of the balloon when it is inflated. For these applications, proximal pharyngeal balloon 30 may optionally have a Shore hardness of between oo-20 and A-30. For other applications, proximal pharyngeal inflatable balloon 30 comprises a material having minimal elasticity as commonly used in medical device cuff technology, e.g., PVC or polyurethane. For these applications, proximal pharyngeal balloon 30 may optionally have a Shore hardness of between OO-30 and A-15.

For some applications, such as in order to provide the ergonomic fit mentioned above, proximal pharyngeal balloon 30, when unconstrained and inflated at a pressure of 5 cm H2O, is shaped such that:
proximal pharyngeal balloon 30 has greatest lateral width W_{B} at a location 77 along proximal pharyngeal balloon 30,
a line segment 85 having greatest lateral width W_{B} extends between opposing lateral sides 83 of proximal pharyngeal balloon 30 at location 77,
proximal pharyngeal balloon 30 has a greatest-width cross-section 91 perpendicular to central longitudinal axis 88 (labeled in FIG. 1D) of laryngeal ventilation support 34, and an outer perimeter 79 of greatest-width cross-section 91 is divided by line segment 85 into a posterior perimeter portion 87A and an anterior perimeter portion 87B, and
greatest-width cross-section 91 is divided by line segment 95 into a posterior cross-sectional portion 89A and an anterior cross-sectional portion 89B.

For some applications:
a ratio of a length of posterior perimeter portion 87A to a length of anterior perimeter portion 87B is between 1.2:1 and 2:1, such as between 1.2:1 and 1.6:1 or between 1.6:1 and 2:1, the lengths measured along the respective perimeter portions,
a ratio of (a) a first greatest distance D_{P}, in greatest-width cross-section 91, between posterior side 78 and line segment 85 to (b) a second greatest distance D_{A}, in greatest-width cross-section 91, between anterior side 76 and line segment 85, is between 1.2:1 and 2:1, such as between 1.2:1 and 1.6:1 or between 1.6:1 and 2:1, and/or
a ratio of an area of posterior cross-sectional portion 89A to an area of anterior cross-sectional portion 89B is between 1.2:1 and 2:1, such as between 1.2:1 and 1.6:1 or between 1.6:1 and 2:1.

For some applications, distal esophageal balloon 32 comprises a highly elastic material, such as silicone, latex, or TPE. For these applications, distal esophageal balloon 32 may optionally have a Shore hardness of between oo-20 and A-30. For other applications, proximal pharyngeal inflatable balloon 30 comprises a material having minimal elasticity as commonly used in medical device cuff technology, e.g., PVC or polyurethane. For these applications, distal esophageal balloon 32 may optionally have a Shore hardness of between OO-30 and A-15.

For some applications, lateral sides 44 of laryngeal ventilation support 34 are shaped so as to define respective wings 86, which are configured to flex toward a central longitudinal axis 88 (labeled in FIG. 1D) of laryngeal ventilation support 34 when squeezed by the laryngeal soft tissue when laryngeal ventilation support 34 is disposed at insertion location 40. Wings 86 may help laryngeal ventilation support 34 accommodate differences in anatomy between patients.

For some applications, proximal pharyngeal balloon 30, when unconstrained and inflated at a pressure of 5 cc H2O, has a greatest lateral distance D_{BL} from a lateral edge of shaft 50 (labeled in FIG. 1D) that is between 5 and 10 mm, and has a greatest anterior-posterior distance D_{BAP} from shaft 50 (labeled in FIG. 1C) that is between 0 and 5 mm.

For some applications, proximal pharyngeal balloon 30 is generally ellipsoidal when unconstrained and inflated at a pressure of 5 cc H2O. This shape may help reduce folds in the proximal pharyngeal balloon that might create air leak around the balloon.

For some applications, laryngeal ventilation support 34 comprises a material having a Shore hardness of between A30 and A90, e.g., between A40 and A80.

Typically, proximal pharyngeal inflatable balloon 30 and distal esophageal inflatable balloon 32 are connected in fluid communication by a tube 33 (labeled in FIGS. 3A-B).

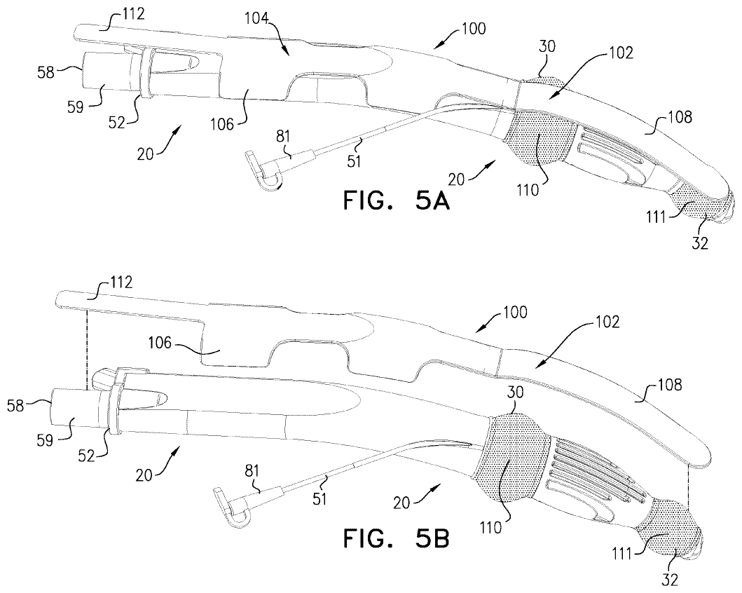

Dual-balloon ventilation tube 20 further comprises an externally-accessible inflation tube 51, for supplying air to and extracting air from proximal pharyngeal inflatable balloon 30 and distal esophageal inflatable balloon 32. Inflation tube 51 has (a) a proximal end 53 that is configured to be disposed outside the patient's oral cavity 24 when laryngeal ventilation support 34 is disposed at insertion location 40, and (b) a distal end that is coupled in fluid communication with an interior of proximal pharyngeal inflatable balloon 30, for supplying air to the balloon, and, typically, for supplying air indirectly to distal esophageal inflatable balloon 32 via tube 33. (For clarity of illustration, inflation tube 51 is shown only in FIGS. 5A-B, although it is provided in all configurations.)

For some applications, inflation tube 51 comprises an inflation tube proximal port connector that comprises a male conical fitting with a taper. For some applications, the taper is at least a 5% taper. For some applications, the taper is a 6% taper, and the male conical fitting with the 6% taper complies with International Standard ISO 594-1:1986, which is the standard for connections to conventional inflation lumen proximal ports of laryngeal tubes.

For some applications, inflation tube 51 comprises an inflation valve 81 that is configured to be disposed outside the patient's oral cavity 24 when laryngeal ventilation support 34 is disposed at insertion location 40, for holding a given inflation of proximal pharyngeal inflatable balloon 30 and distal esophageal inflatable balloon 32. Optionally, inflation valve 81 comprises a check-valve.

Typically, a distal portion of shaft 50 extends into an interior of proximal pharyngeal inflatable balloon 30, as can be seen in FIGS. 2A-B and 3A-B, such that a portion of the external surface of shaft 50 is in fluid communication with an interior of proximal pharyngeal inflatable balloon 30. The distal portion of shaft 50 typically passes through at least a portion, such as all, of proximal pharyngeal inflatable balloon 30, such that a portion of proximal pharyngeal inflatable balloon 30 surrounds 360 degrees of the distal portion of shaft 50.

For some applications, proximal pharyngeal inflatable balloon 30, when filled with air at a pressure of 40 cm H2O and unconstrained, has a volume of more than 1.5 times the volume at a pressure of 5 cm H2O.

For some applications, distal esophageal inflatable balloon 32, when filled with air at a pressure of 40 cm H2O and unconstrained, has a volume of more than 1.5 times the volume at a pressure of 5 cm H2O.

As used in the present application, including in the claims, “unconstrained” means not constrained by the patient's anatomy, a delivery tool, or anything else.

In some applications of the present invention, such as shown in the figures, dual-balloon ventilation tube 20 further comprises a drainage tube that (a) extends from a gastric channel that passes through distal esophageal balloon 32, (a) has a distal gastric opening 92 distal to distal esophageal balloon 32, and (c) has a proximal drainage port at a location outside the patient's oral cavity 24 when laryngeal ventilation support 34 is disposed at insertion location 40. The drainage tube enables extraction and external removal of gastric-discharge products from esophagus 66. The drainage tube is not an essential element of dual-balloon ventilation tube 20, and is not provided in some embodiments of the invention. Therefore, unless specifically stated to the contrary, all features of dual-balloon ventilation tube 20 described herein apply to designs both with and without the drainage tube.

Reference is again made to FIG. 1D. For some applications, respective portions 72A and 72B of a perimeter 74 (labeled in FIG. 1D) of laryngeal ventilation support 34 defined by lateral sides 44 are reflectionally symmetric with each other, typically about a plane of symmetry that extends in an anterior/posterior direction (when laryngeal ventilation support 34 is disposed at insertion location 40, the plane of symmetry corresponds to the sagittal plane of the patient). Because of this reflectional symmetry, laryngeal ventilation support 34 provides balanced support on both sides of pharynx 64 to prevent side-to-side tilting of dual-balloon ventilation tube 20.

Reference is still made to FIG. 1D. For some applications, central longitudinal axis 88 of dual-balloon ventilation tube 20 is defined by a line that passes through a centroid of distal esophageal balloon 32 and a centroid of proximal pharyngeal inflatable balloon 30. A projection of laryngeal ventilation support 34 onto a plane (illustrated in FIG. 1D), in which central longitudinal axis 88 falls, is divided by central longitudinal axis 88 into two lateral sections on either side of central longitudinal axis 88. Typically, an area of one of the lateral sections includes between 40% and 50% of a total area of the projection, such as between 45% and 50% of the total area. Because of this generally even lateral distribution of laryngeal ventilation support 34, laryngeal ventilation support 34 provides balanced support on both sides of pharynx 64 to prevent side-to-side tilting of dual-balloon ventilation tube 20.

Reference is again made to FIG. 4. In some applications of the present invention, a method is provided that comprises inserting distal esophageal inflatable balloon 32, laryngeal ventilation support 34, and proximal pharyngeal inflatable balloon 30 of dual-balloon ventilation tube 20 through anterior opening 22 of oral cavity 24, such that:
proximal end 52 of shaft 50 is disposed outside oral cavity 24,
ventilation port 36 of laryngeal ventilation support 34 is in fluid communication with the patient's laryngeal inlet 38, and laryngeal ventilation support 34 is disposed at insertion location 40 within the patient's pharynx 64 such that both of lateral sides 44 of laryngeal ventilation support 34 contact laryngeal soft tissue of pharynx 64 so as to provide lateral support that centers and inhibits tilting of laryngeal ventilation support 34, such that ventilation port 36 faces anteriorly,
proximal end 58 of airway tube 56 is disposed outside the patient's oral cavity 24,
proximal pharyngeal inflatable balloon 30 makes an air-tight seal with the patient's pharynx 64, and
distal esophageal inflatable balloon 32 makes an air-tight seal with the patient's esophagus 66.

The method further comprises ventilating lungs of the patient using dual-balloon ventilation tube 20.

Reference is still made to FIG. 4. In typical use, dual-balloon ventilation tube 20 should be inserted into a throat while proximal pharyngeal inflatable balloon 30 and distal esophageal inflatable balloon 32 are deflated; however, in practice, laryngeal tubes are often inserted while inflated at ambient pressure. Dual-balloon ventilation tube 20 is typically used for ventilating lungs of the patient.

For some applications, dual-balloon ventilation tube 20 is configured such that when laryngeal ventilation support 34 is disposed at insertion location 40 and proximal pharyngeal balloon 30 is inflated at a pressure of 30 cm H2O, proximal pharyngeal balloon 30 spans both (a) a space between a tongue 97 and a pharyngeal back wall of the throat and (b) a lateral space between left and right tonsils, thereby forming a proximal seal between oral cavity 24 and a pharyngeal space above the glottis, distal to proximal pharyngeal balloon 30.

For some applications, dual-balloon ventilation tube 20 is configured such that when laryngeal ventilation support 34, while inflation valve 81 is closed and the pressure within proximal pharyngeal inflatable balloon 30 equals ambient pressure of ambient air, is inserted from outside oral cavity 24 of the patient to insertion location 40 without opening inflation valve 81, the pressure within proximal pharyngeal inflatable balloon 30 increases from the ambient pressure to an inserted-location pressure, the inserted-location pressure less than 60 cm H2O, such as less than 45 cm H2O, e.g., less than 30 cm H2O.

For some applications, inserting proximal pharyngeal inflatable balloon 30 comprises inserting proximal pharyngeal inflatable balloon 30, while inflation valve 81 is closed, from outside oral cavity 24 until laryngeal ventilation support 34 is disposed at insertion location 40 without opening inflation valve 81. For some applications, dual-balloon ventilation tube 20 is configured such that when proximal pharyngeal inflatable balloon 30, while inflation valve 81 is closed and a pressure within proximal pharyngeal inflatable balloon 30 equals ambient pressure, is inserted from outside oral cavity 24 until laryngeal ventilation support 34 is disposed at insertion location 40 without opening inflation valve 81, the pressure within proximal pharyngeal inflatable balloon 30 increases from the ambient pressure to an inserted-location pressure, the inserted-location pressure less than 60 cm H2O, such as less than 45 cm H2O. e.g., less than 30 cm H2O.

For some applications, inserting proximal pharyngeal inflatable balloon 30 while inflation valve 81 is closed comprises inserting proximal pharyngeal inflatable balloon 30 while inflation valve 81 is closed and the pressure within proximal pharyngeal inflatable balloon 30 equals ambient pressure.

For some applications, inserting proximal pharyngeal inflatable balloon 30 while inflation valve 81 is closed comprises inserting proximal pharyngeal inflatable balloon 30 while inflation valve 81 is closed without deflating proximal pharyngeal inflatable balloon 30 before inserting proximal pharyngeal inflatable balloon 30.

For some applications, the method does not comprise measuring the pressure within proximal pharyngeal inflatable balloon 30. For other applications, the pressure is measured.

For some applications, the method does not comprise adding air to or removing air from proximal pharyngeal inflatable balloon 30 after inserting proximal pharyngeal inflatable balloon 30 until laryngeal ventilation support 34 is disposed at insertion location 40. For other applications, air is added to or removed from proximal pharyngeal inflatable balloon 30 after inserting proximal pharyngeal inflatable balloon 30, such as for applications in which the pressure is measured, as described above, and found to be too low or too high, respectively.

For some applications, the method does not comprise adjusting the pressure within proximal pharyngeal inflatable balloon 30 after inserting proximal pharyngeal inflatable balloon 30 until laryngeal ventilation support 34 is disposed at insertion location 40. For other applications, the pressure is adjusted within proximal pharyngeal inflatable balloon 30 after inserting proximal pharyngeal inflatable balloon 30 until laryngeal ventilation support 34 is disposed at insertion location 40, such as for applications in which the pressure is measured, as described above, and found to be too low or too high.

For some applications, dual-balloon ventilation tube 20 is configured such that when a volume of 6 cc of ambient air, the volume measured at atmospheric pressure, is introduced into externally-accessible inflation tube 51 while proximal pharyngeal balloon 30 and distal esophageal inflatable balloon 32 are unconstrained, disposed in ambient air that is at standard atmospheric pressure (i.e., air pressure at sea level), and at is an inflation pressure of 30 cm H2O:
the inflation pressure in proximal pharyngeal balloon 30, measured one minute after introduction of the volume of 6 cc of the ambient air, is less than 40 cm H2O.

(Waiting the one minute allows the pressure in proximal pharyngeal balloon 30 to stabilize.)

This relatively low pressure increase upon moderate additional inflation reduces the risk of damage to soft tissue that might be caused by higher pressures within proximal pharyngeal balloon 30.

In an experiment conducted on behalf of the inventor, a dual-balloon ventilation tube similar to dual-balloon ventilation tube 20 was compared to a Ambu® King LTS-D™ Disposable Laryngeal Tube size #4 (Ambu A/S, Ballerup, Denmark). The respective proximal balloons were inflated to an inflation pressure of 30 cm H2O. 6 cc of ambient air was drawn into a syringe and introduced into each of the respective proximal balloons. The inflation pressure in the proximal balloon of the dual-balloon ventilation tube similar to dual-balloon ventilation tube 20 increased from the initial 30 cm H20 to 36.4 cm H2O, while the inflation pressure in the proximal balloon of the Ambu® King device increased from the initial 30 cm H2O to 43.9 cm H2O.

For some applications, distal esophageal inflatable balloon 32 has one or more of the pressure-related properties of proximal pharyngeal inflatable balloon 30 described above, mutatis mutandis.

Reference is now made to FIGS. 5A-B, which are schematic illustrations of an insertion posterior protector 100 coupled to and decoupled from dual-balloon ventilation tube 20, respectively, in accordance with an application of the present invention. Insertion posterior protector 100 assists with insertion of dual-balloon ventilation tube 20 into the patient.

Insertion posterior protector 100 comprises an elongate sheet of material 102 that is shaped so as to define:
a proximal coupling portion 104, which is shaped so as to define one or more couplers 106 that are configured to removably couple the proximal coupling portion to shaft 50 proximally to proximal pharyngeal balloon 30, and
a distal protection portion 108, which is shaped so as to partially cover a posterior external surface 110 of proximal pharyngeal balloon 30 when proximal coupling portion 104 is removably coupled to shaft 50, so as to protect proximal pharyngeal balloon 30 from puncture (such as by the patient's teeth) during insertion thereof into the patient's oral cavity 24.

Optionally, as shown in FIG. 5A, distal protection portion 108 is shaped so as to additionally partially cover a posterior external surface 111 of distal esophageal balloon 32 when proximal coupling portion 104 is removably coupled to shaft 50, so as to protect distal esophageal balloon 32 from puncture (such as by the patient's teeth) during insertion thereof into the patient's oral cavity 24.

For some applications, the one or more couplers 106 are shaped so as to form snap-on couplings to shaft 50, such as illustrated. For other applications, the one or more couplers 106 are shaped as to otherwise mechanically or chemically removably couple the proximal coupling portion to shaft 50 proximally to proximal pharyngeal balloon 30.

For some applications, elongate sheet of material 102 of insertion posterior protector 100 is further shaped so as to define a proximal handle 112, which is shaped so as to be disposed outside the patient's oral cavity 24 when laryngeal ventilation support 34 is disposed at insertion location 40. Proximal handle 112 may be used to decouple insertion posterior protector 100 from shaft 50 and remove insertion posterior protector 100 from the patient's oral cavity 24 after laryngeal ventilation support 34 has been disposed at insertion location 40.

For some applications, elongate sheet of material 102 has an average thickness of between 0.5 and 2 mm. For some applications, elongate sheet of material 102 comprises a polymer (e.g., PVC, ABS, or polyurethane) or a metal.

In the present application, including in the claims, all pressures are gauge pressures that are zero-referenced against ambient air pressure.

Typically, but not necessarily, as is conventional in the art, proximal pharyngeal inflatable balloon 30 and distal esophageal inflatable balloon 32 are single-layer balloons, i.e., have only a single wall, as can be seen in the cross-sectional figures.

All publications, patent applications, patents, and other references mentioned herein are incorporated by reference in their entirety. In case of conflict, the patent specification, including definitions, will prevail. In addition, the materials, methods, and examples are illustrative only and not intended to be limiting.

In an embodiment, techniques and apparatus described in one or more of the following applications, which are assigned to the assignee of the present application and incorporated herein by reference, are combined with techniques and apparatus described herein:
U.S. Provisional App. No. 62/592,020, filed Nov. 29, 2017
U.S. Pat. No. 10,369,311 to Zachar
U.S. Pat. No. 10,173,022 to Zachar
U.S. Provisional App. No. 62/789,208, filed Jan. 7, 2019
PCT Publication WO 2019/106670 to Zachar
PCT Publication WO 2019/198081 to Zachar
U.S. Pat. No. 10,744,287 to Zachar
U.S. Provisional App. No. 63/035,057, filed Jun. 5, 2020
U.S. Provisional App. No. 63/079,639, filed Sep. 17, 2020
U.S. Provisional App. No. 63/079,628, filed Sep. 17, 2020

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A dual-balloon ventilation tube comprising: a laryngeal ventilation support, which:
is shaped so as to define a ventilation port, which is configured to be in fluid communication with a laryngeal inlet of a patient when the laryngeal ventilation support is disposed at an insertion location within a pharynx of the patient, and has a greatest lateral width of between 2 and 6 cm, measured between lateral sides of the laryngeal ventilation support, such that when the laryngeal ventilation support is disposed at the insertion location, both of the lateral sides contact laryngeal soft tissue of the pharynx so as to provide lateral support that centers and inhibits tilting of the laryngeal ventilation support, such that the ventilation port faces anteriorly;
a shaft having proximal and distal ends, the proximal end configured to be disposed outside an oral cavity of the patient when the laryngeal ventilation support is disposed at the insertion location, and the distal end supporting the laryngeal ventilation support;
an airway tube, which passes through at least a longitudinal portion of the shaft, and has a proximal end that is configured to be disposed outside the patient's oral cavity when the laryngeal ventilation support is disposed at the insertion location, and a distal end that is in fluid communication with the ventilation port;
a proximal pharyngeal inflatable balloon, which surrounds a distal portion of the shaft proximal to the laryngeal ventilation support, and which is configured to make an air-tight seal with a pharynx of the patient when the laryngeal ventilation support is disposed at the insertion location; and
a distal esophageal inflatable balloon, which is disposed distal to the laryngeal ventilation support, and which is configured to make an air-tight seal with an esophagus of the patient when the laryngeal ventilation support is disposed at the insertion location.

2. The dual-balloon ventilation tube according to claim 1, wherein the greatest lateral width of the laryngeal ventilation support is between 3 and 6 cm.

3. The dual-balloon ventilation tube according to claim 1, wherein the laryngeal ventilation support, along a 1-cm-long longitudinal portion thereof, has an average lateral width greater than 90% of the greatest lateral width of the laryngeal ventilation support.

4. The dual-balloon ventilation tube according to claim 1, wherein the greatest lateral width of the laryngeal ventilation support is at least 1.5 times a greatest anterior-posterior height of the laryngeal ventilation support.

5. The dual-balloon ventilation tube according to claim 4, wherein the greatest lateral width of the laryngeal ventilation support is at least 2 times the greatest anterior-posterior height of the laryngeal ventilation support.

6. The dual-balloon ventilation tube according to claim 1, wherein respective portions of a perimeter of the laryngeal ventilation support defined by the lateral sides are reflectionally symmetric with each other.

7. The dual-balloon ventilation tube according to claim 1,
wherein a central longitudinal axis of the dual-balloon ventilation tube is defined by a line that passes through a centroid of the distal esophageal balloon and a centroid of the proximal pharyngeal inflatable balloon,
wherein a projection of the laryngeal ventilation support onto a plane, in which the central longitudinal axis falls, is divided by the central longitudinal axis into two lateral sections on either side of the central longitudinal axis 88, and
wherein an area of one of the lateral sections includes between 40% and 50% of a total area of the projection.

8. The dual-balloon ventilation tube according to claim 7, wherein the area of the one of the lateral sections includes between 45% and 50% of the total area of the projection.

9. The dual-balloon ventilation tube according to claim 1, wherein the laryngeal ventilation support is shaped so as to define an anterior side that is configured to stabilize the laryngeal ventilation support with respect to the laryngeal inlet when the laryngeal ventilation support is disposed at the insertion location.

10. The dual-balloon ventilation tube according to claim 1, wherein the proximal pharyngeal balloon, when unconstrained and inflated at a pressure of 5 cm H2O, has a greatest lateral width that is between 1.2 and 2 times the greatest lateral width of the laryngeal ventilation support.

11. The dual-balloon ventilation tube according to claim 1, wherein the proximal pharyngeal balloon, when unconstrained and inflated at a pressure of 5 cm H2O, has a greatest anterior-posterior height that is between 1 and 2 times a greatest anterior-posterior height of the laryngeal ventilation support.

12. The dual-balloon ventilation tube according to claim 1, wherein the proximal pharyngeal balloon has a higher-pressure aspect ratio equal to a greatest lateral width of the proximal pharyngeal balloon divided by a greatest anterior-posterior height of the proximal pharyngeal balloon when the proximal pharyngeal balloon is unconstrained and inflated at a pressure of 30 cm H2O,
wherein the proximal pharyngeal balloon has a lower-pressure aspect ratio equal to a greatest lateral width of the proximal pharyngeal balloon divided by a greatest anterior-posterior height of the proximal pharyngeal balloon when the proximal pharyngeal balloon is unconstrained and inflated at a pressure of 5 cm H2O, and wherein the higher-pressure aspect ratio is less than the lower-pressure aspect ratio.

13. The dual-balloon ventilation tube according to claim 12, wherein the higher-pressure aspect ratio is less than 85% of the lower-pressure aspect ratio.

14. The dual-balloon ventilation tube according to claim 1, wherein the proximal pharyngeal balloon, when unconstrained and inflated at a pressure of 5 cc H2O, has a greatest lateral width that is between 1.2 and 2 times a lateral width of the shaft at an interface between the shaft and a proximal end of the proximal pharyngeal balloon.

15. The dual-balloon ventilation tube according to claim 1, wherein the proximal pharyngeal balloon, when unconstrained and inflated at a pressure of 5 cc H2O, has a greatest anterior-posterior distance from the shaft that is between 0 and 5 mm.

16. The dual-balloon ventilation tube according to claim 1, wherein the proximal pharyngeal balloon, when unconstrained and inflated at a pressure of 5 cc H2O, has:
a greatest lateral width that is between 1.2 and 2 times a lateral width of the shaft at an interface between the shaft and a proximal end of the proximal pharyngeal balloon, and
a greatest anterior-posterior distance from the shaft that is between 0 and 5 mm.

17. The dual-balloon ventilation tube according to claim 1,
wherein the proximal pharyngeal balloon, when unconstrained and inflated at a pressure of 5 cc H2O, has a greatest lateral distance from a lateral edge of the shaft that is between 5 and 10 mm, and has a greatest anterior-posterior distance from the shaft that is between 0 and 5 mm.

18. The dual-balloon ventilation tube according to claim 1, wherein the lateral sides of the laryngeal ventilation support are shaped so as to define respective wings, which are configured to flex toward a central longitudinal axis of the laryngeal ventilation support when squeezed by the laryngeal soft tissue when the laryngeal ventilation support is disposed at the insertion location.

19. The dual-balloon ventilation tube according to claim 1, wherein the proximal pharyngeal balloon is generally ellipsoidal when unconstrained and inflated at a pressure of 5 cc H2O.

20. The dual-balloon ventilation tube according to claim 1, wherein the laryngeal ventilation support comprises a material having a Shore hardness of between A30 and A90.

21-58. (canceled)
